(19) United States
(12) Patent Application Publication
(10) Pub. No.: US 2010/0041732 A1
(43) Pub. Date: Feb. 18, 2010

(54) METHODS FOR IDENTIFYING COMPOUNDS THAT INHIBIT HIV INFECTION

(75) Inventors: Jeremy Caldwell, La Jolla, CA (US); Deborah Nguyen, San Diego, CA (US); Kelly Kuhen, Solana Beach, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 11/815,311

(22) PCT Filed: Feb. 6, 2006
(86) PCT No.: PCT/US06/04264

§ 371 Date: Jun. 24, 2008

Related U.S. Application Data
(60) Provisional application No. 60/650,789, filed on Feb. 7, 2005.

(51) Int. Cl. G01N 33/53; C12Q 1/48; A61K 31/7088; A61P 31/18

(52) U.S. Cl. 514/44 A; 435/15; 435/7.92

(57) ABSTRACT

This invention provides novel methods for identifying agents that inhibit HIV infection. The anti-HIV agents are identified by screening test compounds for ability to down-regulate the kinase activity or the expression of a PAK3 molecule. Such PAK3 modulators can be further examined for their activity in inhibiting HIV infection. These novel anti-HIV agents are useful in the prevention or treatment of HIV infection and conditions associated with HIV infection.

### CROSS-REFERENCE TO RELATED APPLICATIONS

This application claims the benefit of priority under 35 U.S.C. §119(e) to U.S. Provisional Patent Application No. 60/650,789, filed Feb. 7, 2005. The disclosure of the priority application is incorporated herein by reference in its entirety and for all purposes.

### FIELD OF THE INVENTION

The present invention generally relates to methods for identifying compounds that inhibit HIV infection and therapeutic applications of such compounds. More particularly, the invention pertains to screening for inhibitors of PAK3 kinase, and to methods of using such inhibitors to treat or prevent HIV infections.

### BACKGROUND OF THE INVENTION

Human immunodeficiency viruses (HIV) are lentiviruses from the family of retroviridae. It was estimated that transmission of HIV through sexual contact and during pregnancy accounts for up to 90% of AIDS cases worldwide. This transmission is initiated by the passage of HIV across the mucosal barrier of sexual organs or placenta when exposed to infectious body fluids such as semen, vaginal secretions, or blood. The remaining AIDS cases are due to the transfusion of HIV-contaminated blood, needle sharing among intravenous drug users, accidental exposure to HIV-contaminated body fluids during invasive procedures, and other situations wherein infectious virus can come into direct contact with susceptible human tissues.

Effective compounds with anti-HIV activity that could be used to prevent and/or treat AIDS are still lacking. Toxicity or undesirable side effects of the common drugs for treating HIV infection, e.g., AZT or HIV protease inhibitors, are incompatible with their antiviral activity when used at an effective pharmaceutical concentration. There is still a need in the art for better alternative compounds for preventing and treating AIDS and HIV infection. The instant invention addresses this and other needs.

### SUMMARY OF THE INVENTION

In one aspect, the present invention provides methods for identifying agents that inhibit HIV infection. The methods entail assaying a biological activity of a p21-activated kinase 3 (PAK3) molecule in the presence of test compounds to identify compounds that inhibit the biological activity of the PAK3 molecule. These methods can further include testing the identified compound for ability to inhibit HIV infection. In some methods, the assayed biological activity is the kinase activity of the PAK3 molecule. It can also be the expression of a gene encoding the PAK3 molecule. Typically, the PAK3 molecule employed in the screening is derived from a mammalian cell. In some preferred embodiments, a human PAK3 molecule is employed.

In some methods, the ability to inhibit HIV infection by the Pak3-modulating compounds is examined by monitoring expression of a reporter gene under the control of the HIV LTR promoter in HIV-infected cells. In some of the methods, the cells employed are HeLa-CD4-Bgal cells. In some of the methods, the reporter gene employed is a beta-galactosidase gene.

In some other methods, the ability to inhibit HIV infection by the Pak3-modulating compounds is examined by monitoring HIV replication in PAK3-expressing cells in vitro. In some of these methods, HIV replication is monitored via a p24 antigen ELISA assay or a reverse transcriptase activity assay. In some of the methods, primary macrophage cells are used.

In a related aspect, the invention provides methods for identifying agents that inhibit HIV infection. These methods involve (a) screening test compounds to identify PAK3-modulating compounds that down-regulate the kinase activity or cellular level of a PAK3 molecule, and (b) testing the identified PAK3-modulating compounds for ability to inhibit HIV infection. Preferably, the PAK3 molecule used in the methods is derived from a mammalian cell. In some methods, a human PAK3 molecule is employed.

In some of these methods, the ability to inhibit HIV infection by the PAK3-modulating compounds is examined by monitoring expression of a reporter gene under the control of HIV LTR promoter in HIV-infected cells. In some of the methods, the cells employed in the methods are, e.g., HeLa-CD4-Bgal cells. In some the methods, the reporter gene is a beta-galactosidase gene.

In some other methods, the ability to inhibit HIV infection by the PAK3-modulating compounds is examined by monitoring HIV replication in PAK3-expressing cells in vitro. For example, HIV replication can be monitored via a p24 antigen ELISA assay or a reverse transcriptase activity assay. In some methods, primary macrophage cells are used in the screening.

In another aspect, the present invention provides methods for treating HIV infection in a subject. The methods entail administering to the subject a pharmaceutical composition that contains an effective amount of a PAK3-modulating compound. The PAK3-modulating compound can down-regulate the kinase activity or the expression of a PAK3 molecule. In some of these methods, the PAK3-modulating compound down-regulates the activity of a human PAK3. In some methods, the PAK3-modulating compound employed can inhibit HIV replication in a PAK3-expressing cell in vitro. In some of the methods, the PAK3-modulating compound is administered to the subject concurrently with a known anti-HIV drug.

A further understanding of the nature and advantages of the present invention may be realized by reference to the remaining portions of the specification and claims.

### BRIEF DESCRIPTION OF THE DRAWINGS

FIG. 1 shows schematically the siRNA screening which identified that PAK3 plays a role in HIV infection.

FIG. 2 shows that knockdown of PAK3 via unique siRNA independently inhibits HIVIIIb infection in the HeLa-T4-Bgal cell line.

### DETAILED DESCRIPTION

### I. Overview

The invention is predicated in part on the discovery by the present inventors that p21-activated kinase 3 (PAK3) is involved in HIV infection. The present inventors performed a sub-genomic siRNA screen for host proteins that are involved in HIV infection by monitoring expression of a reporter gene under the control of HIV LTR promoter in HeLaCD4Bgal cells. The data showed that inhibiting PAK3 expression had a negative impact on HIV infection. Utilizing validated siRNA, the inventors observed that inhibition of PAK3 or PAK1 expression using siRNA significantly inhibited HIV infection while PAK2 depletion had no effect. These findings indicate that, in addition to PAK1, PAK3 could also play an important role in enhancing HIV infection.

In accordance with these discoveries, the present invention provides methods for screening novel agents that inhibit HIV infection. Test compounds are first screened for ability to modulate a biological activity of a PAK3 molecule, e.g., its expression or its kinase activity. The agents thus identified are then typically further tested for ability to modulate HIV infection or an activity indicating HIV infection. Various PAK3 molecules can be employed in the screening assays. For example, PAK3 from human, rat or mouse can be used to screen for the modulators. In some preferred embodiments, a human PAK3 molecule is used.

The methods of the present invention also find therapeutic applications. Pharmacological inhibition of PAK3 provides a novel approach for treating or preventing conditions related to HIV infection. Typically, the approach entails administering to a subject a PAK3 inhibitor that can be identified in accordance with the present invention.

The following sections provide guidance for making and using the compositions of the invention, and for carrying out the methods of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Oxford Dictionary of Biochemistry and Molecular Biology, Smith et al. (eds.), Oxford University Press (revised ed., 2000); Dictionary of Microbiology and Molecular Biology, Singleton et al. (Eds.), John Wiley & Sons (3^{rd} ed., 2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

HIV refers to human immunodeficiency virus (HIV) family of retroviruses. These viruses includes, but not limited to, HIV-I, HIV-II, HIV-III (also known as HTLV-I, LAV-1, LAV-2), and the like. As used herein, HIV can be any strains, forms, subtypes, clades and variations in the HIV family.

The terms “identical” or “sequence identity” in the context of two nucleic acid sequences or amino acid sequences refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment. In one class of embodiments, the polypeptides herein are at least 70%, generally at least 75%, optionally at least 80%, 85%, 90%, 95% or 99% or more identical to a reference polypeptide, as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 95%, 99% or more identical to a reference nucleic acid, as measured by BLASTN (or CLUSTAL, or any other available alignment software) using default parameters.

The terms “substantially identical” nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “modulate” with respect to biological activities of a PAK3 molecule refers to a change in the cellular level, subcellular localization or other biological activities of PAK3 (e.g., its kinase activity). Modulation of PAK3 activities can be up-regulation (i.e., activation or stimulation) or down-regulation (i.e. inhibition or suppression). For example, modulation may cause a change in cellular level or enzymatic modification (e.g., phosphorylation) of PAK3, binding characteristics (e.g., binding to a substrate or ATP), or any other biological, functional, or immunological properties of PAK3 proteins. The change in activity can arise from, for example, an increase or decrease in expression of a PAK3-encoding gene, the stability of mRNA that encodes the PAK3 protein, translation efficiency, or from a change in other bioactivities of the PAK3 enzymes (e.g., its kinase activity). The mode of action of a PAK3 modulator can be direct, e.g., through binding to the PAK3 protein or to a gene encoding the PAK3 protein. The change can also be indirect, e.g., through binding to and/or modifying (e.g., enzymatically) another molecule which otherwise modulates PAK3.

The term “subject” refers to mammals, particularly humans. It encompasses other non-human animals such as cows, horses, sheep, pigs, cats, dogs, mice, rats, rabbits, guinea pigs, monkeys.

A “variant” of a molecule such as a PAK3 is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

### III. Screening Scheme

PAK kinases exist as homodimers in the inactive state, with the autoinhibitory domain of the protein preventing activity. In vitro, PAKs bind to and are activated by the small GTP-binding proteins of the Rho family, Cdc42 and Rac. These GTPases bind to a region of the N-terminal regulatory domain (the p21-binding domain or Cdc42- and Rac-interacting binding motif) of the PAKs. As a result of the binding, the PAKs release an N-terminal inhibitory domain and allow autophosphorylation that leads to full kinase activity. There are at least 7 p21-activated protein kinases. PAK3 is a member of the Group I PAK kinases, which also includes PAK1 and PAK2. PAK1, PAK2, and PAK3 are respectively also termed p68PAK1/αPAK, p62PAK2/γPAK and p65PAK3/βPAK. These three kinases have 93% homology in their kinase domains and differing mainly in their tissue distribution. While PAK2 is ubiquitously expressed, PAK1 is more restricted and PAK3 is predominantly found in the brain. In addition to the kinase cascades, the Group I PAKs have been shown to be involved in cytoskeletal organization, cell morphology and motility, neurogenesis, cancer metastasis, and apoptosis.

According to the present invention, novel inhibitors of HIV infection are identified by first screening test compounds for ability to modulate (e.g., inhibit) a biological activity of PAK3. The biological activity of PAK3 to be monitored in the screening assays can be its kinase activity. It can also be its expression or its cellular level, as well as a specific binding of PAK3 to a test compound. In some methods, test compounds are screened to identify PAK3 selective modulators that do not have significant activity on one or more of the other PAK kinases (e.g., PAKs 3-7). After test compounds that modulate a biological activity of PAK3 have been identified, they are typically further examined for ability to modulate HIV infection or an activity indicating HIV infection. This step serves to confirm that by modulating the biological activity of PAK3, compounds identified in the first step can indeed regulate (e.g., inhibit) HIV infection. These methods can also additionally include a control step to examine effect of the compounds on HIV infection in cells that do not express PAK3.

Various biochemical and molecular biology techniques or assays well known in the art can be employed to screen for PAK3 modulators. Such techniques are described in, e.g., Handbook of Drug Screening, Seethala et al. (eds.), Marcel Dekker (1^{st} ed., 2001); High Throughput Screening: Methods and Protocols (Methods in Molecular Biology, 190), Janzen (ed.), Humana Press (1^{st} ed., 2002); Current Protocols in Immnunology, Coligan et al. (Ed.), John Wiley & Sons Inc (2002); Sambrook et al., Molecular Cloning: A Laboratory Manual, Cold Spring Harbor Press (3^{rd} ed., 2001); and Brent et al., Current Protocols in Molecular Biology, John Wiley & Sons, Inc. (ringbou ed., 2003).

PAK3 from various species can be employed in screening the PAK3 modulators of the present invention. Preferably, a mammalian cell-derived PAK3 molecule is employed. Examples include PAK3 molecules encoded by polynucleotides with accession numbers NM_{—}002578, AF068864 and AB102659 (human); AB102660 and AB102661 (chimpanzee or ape); NM_{—}008778, AJ496262 and AJ496263 (mouse); and NM_{—}019210 (rat). Preferably, a human PAK3 molecule is used. Examples of PAK3 polypeptide sequences include amino acid sequences with Accession Nos. NP_{—}002569, Q75914, AAF67008, AAC36097, AAC52354, and BAC81128 (human PAK3); Accession Nos. BAC81129 and BAC81130 (chimpanzee or ape); Accession Nos. AAH53403, I49376, CAD42791, CAD42790 and Q61036 (mouse PAK3); and Accession No. Q62829 (rat PAK3). These molecules have all been biochemically characterized in the art. Any of these PAK3 sequences or substantially identical sequences thereof can be employed in the screening assay to identify PAK3 modulators in the present invention. Similarly, sequences of the other PAK enzymes (e.g., PAKs 4-7) from human and other species are also known in the art (e.g., Accession Nos. NM_{—}005884, NM_{—}027470, AB040812, AJ277826, NM_{—}020168, XM_{—}111790, NM_{—}020341, and NM_{—}172858).

Methods for isolating PAK3 sequences and other PAK sequences, as well as expressing and purifying these enzymes are also described in the art. See, e.g., McPhie et al., J Neurosci. 23(17):6914-27, 2003; Rousseau et al., J Biol Chem. 278(6):3912-20, 2003; and Hashimoto et al., J Biol Chem. 276(8):6037-45, 2001; Sells et al., Curr Biol. 7(3):202-10, 1997; and Goeckeler et al., J Biol Chem. 275(24):18366-74, 2000; Cau et al., J. Cell Biol. 155:1029-1042, 2001; Pandey et al., Oncogene 21:3939-3948, 2002; Lu et al., J. Biol. Chem. 278:10374-10380, 2003; and Barac et al., J. Biol. Chem. 279:6182-6189, 2004.

In addition to an intact PAK3 molecule or nucleic acid encoding the intact PAK3 molecule, a PAK3 fragment (e.g., the kinase domain), analog, or a functional derivative can also be used. The PAK3 fragments that can be employed in these assays usually retain one or more of the biological activities of the PAK3 molecule (typically, its kinase activity). As noted above, PAK3s from the different species have already been sequenced and well characterized. Therefore, their fragments, analogs, derivatives, or fusion proteins can be easily obtained using methods well known in the art. For example, a functional derivative of a PAK3 can be prepared from a naturally occurring or recombinantly expressed protein by proteolytic cleavage followed by conventional purification procedures known to those skilled in the art. Alternatively, the functional derivative can be produced by recombinant DNA technology by expressing only fragments of a PAK3 that retain its kinase activity.

### IV. Test Compounds

Test agents that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecules, e.g., molecules with a molecular weight of not more than about 500 or 1,000. Preferably, high throughput assays are adapted and used to screen for such small molecules. In some methods, combinatorial libraries of small molecule test agents as described above can be readily employed to screen for small molecule modulators of PAK3s. A number of assays are available for such screening, e.g., as described in Schultz et al., Bioorg Med Chem Lett 8:2409-2414, 1998; Weller et al., Mol Divers. 3:61-70, 1997; Fernandes et al., Curr Opin Chem Biol 2:597-603, 1998; and Sittampalam et al., Curr Opin Chem Biol 1:384-91, 1997.

Libraries of test agents to be screened with the claimed methods can also be generated based on structural studies of the PAK3 polypeptides, their fragments or analogs. Such structural studies allow the identification of test agents that are more likely to bind to the PAK3 polypeptides. The three-dimensional structure of a PAK3 polypeptide can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of a PAK3 polypeptide structure provides another means for designing test agents for screening PAK3 modulators. Methods of molecular modeling have been described in the literature, e.g., U.S. Pat. No. 5,612,894 entitled “System and method for molecular modeling utilizing a sensitivity factor”, and U.S. Pat. No. 5,583,973 entitled “Molecular modeling method and system”. In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wuthrich (Wiley-Interscience, New York 1986).

Modulators of the present invention also include antibodies that specifically bind to a PAK3 polypeptide. Such antibodies can be monoclonal or polyclonal. Such antibodies can be generated using methods well known in the art. For example, the production of non-human monoclonal antibodies, e.g., murine or rat, can be accomplished by, for example, immunizing the animal with a PAK3 polypeptide or its fragment (See Harlow & Lane, Antibodies, A Laboratory Manual, Cold Spring Harbor Laboratory Press, New York, 1988). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to a PAK3 polypeptide of the present invention.

Human antibodies against a PAK3 polypeptide can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using a PAK3 polypeptide or its fragment.

### V. Screen Test Compounds for PAK3 Modulators

To identify novel compounds that inhibit HIV infection, test compounds are first screened for ability to modulate a biological activity of PAK3 as described herein. In some preferred embodiments, test compounds are examined for ability to modulate (e.g., inhibit) the kinase activity of PAK3. Effect of test compounds on the kinase activity of PAK3 as well as other PAKs (e.g., PAKs 4-7) can be monitored using kinase assays well known in the art. See, e.g., King et al., Nature 396:180-3, 1998; Joneson et al., J Biol Chem 273: 7743-7749, 1998; Chiloeches et al., Mol Cell Biol. 21(7):2423-34, 2001; Hashimoto et al., J Biol Chem. 276(8):6037-45, 2001; Rousseau et al., J Biol Chem. 278(6):3912-20, 2003; McPhie et al., J Neurosci. 23(17):6914-27, 2003; Rashid et al., J Biol Chem. 276(52):49043-52, 2001; and Goeckeler et al., J Biol Chem. 275(24):18366-74, 2000. For example, kinase activity of a PAK molecule (e.g., PAK3) can be examined in vitro by measuring autophosphorylation of a purified PAK polypeptide or a PAK protein precipitated from cell lysate (e.g., lysate of neuron cells). Alternatively, other than monitoring its autophosphorylation, test compounds can also be screened for ability to modulate phosphorylation of another substrate by a PAK kinase. Examples of PAK3 substrates include Raf-1, H2B or MBP (myelin basic protein) as described in the art, e.g., Rousseau et al., J Biol Chem. 278:3912-20, 2003; and King et al., Nature 396:180-3, 1998.

In some embodiments, test compounds can be first screened for their ability to bind to a PAK3 polypeptide. Compounds thus identified can be further subject to assay for ability to modulate (e.g., to inhibit) PAK3 kinase activity as described above. Binding of test agents to a PAK3 polypeptide can be assayed by a number of methods including, e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The test agent can be identified by detecting a direct binding to the PAK3 polypeptide, e.g., co-immunoprecipitation with the PAK3 polypeptide by an antibody directed to the PAK3 polypeptide. The test agent can also be identified by detecting a signal that indicates that the agent binds to the PAK3 polypeptide, e.g., fluorescence quenching or fluorescence polarization.

In some other methods, test agents are assayed for activity to modulate expression or cellular level of PAK3, e.g., transcription, translation, or post-translational modification. Various biochemical and molecular biology techniques well known in the art can be employed to practice the present invention. Such techniques are described in, e.g., Sambrook et al., Molecular Cloning: A Laboratory Manual, Cold Spring Harbor Press, N.Y., Second (1989) and Third (2000) Editions; and Ausubel et al., Current Protocols in Molecular Biology, John Wiley & Sons, Inc., New York (1987-1999). In some embodiments, endogenous levels of a PAK3 kinase can be directly monitored in cells normally expressing PAK3 (e.g., neuron cells). In some embodiments, expression or cellular level of a PAK3 can be examined in an expression system using cloned cDNA or genomic sequence encoding the PAK3.

Alternatively, modulation of expression of a PAK3 gene can be examined in a cell-based system by transient or stable transfection of an expression vector into cultured cell lines. Assay vectors bearing transcription regulatory sequences (e.g., promoter) of a PAK3 gene operably linked to reporter genes can be transfected into any mammalian host cell line for assays of promoter activity. Constructs containing a PAK3 gene (or a transcription regulatory element of a PAK3 gene) operably linked to a reporter gene can be prepared using only routinely practiced techniques and methods of molecular biology (see, e.g., Sambrook et al. and Ausubel et al., supra). General methods of cell culture, transfection, and reporter gene assay have been described in the art, e.g., Ausubel, supra; and Transfection Guide, Promega Corporation, Madison, Wis. (1998). Any readily transfectable mammalian cell line may be used to assay PAK3 promoter function or to express PAK3, e.g., CHO, COS, HCT116, HEK 293, MCF-7, and HepG2 are all suitable cell lines.

When inserted into the appropriate host cell, the transcription regulatory elements in the expression vector induces transcription of the reporter gene by host RNA polymerases. Reporter genes typically encode polypeptides with an easily assayed enzymatic activity that is naturally absent from the host cell. Typical reporter polypeptides for eukaryotic promoters include, e.g., chloramphenicol acetyltransferase (CAT), firefly or Renilla luciferase, beta-galactosidase, beta-glucuronidase, alkaline phosphatase, and green fluorescent protein (GFP).

Some of the screening methods are directed to identifying agents that selectively modulate PAK3 over one or more of the other PAK kinases (e.g., PAK1, PAK2, and PAKs 4-7). For example, in some methods, the test compounds are screened for PAK3 modulators that do not significantly affect the kinase activity of PAK4 or PAK7. In these methods, after identifying PAK3-modulating compounds (e.g., compounds that modulate PAK3 kinase activity), the identified compounds are further examined for any effect on the kinase activity of PAK4 or PAK7. If measured by its ability to inhibit the expression or the kinase activity of a PAK kinase, a PAK3-selective compound typically has an IC₅₀ (effective concentration that causes 50% of the maximum inhibition) for a PAK3 molecule from a species (e.g., human, mouse, or rat) that is at least 2, 5, 10, 25, 50, 100, 500, or 1000 fold lower than its IC₅₀ for the PAK4 enzyme and/or its IC₅₀ for the PAK7 enzyme from the same species. If selectivity is measured by its binding to the PAK enzymes, a PAK3-selective compound usually has a binding specificity that is at least 2, 5, 10, 25, 50, or 100 fold higher than its binding affinity for PAK4 or PAK7.

### VI. Screen for HIV-Inhibiting Compounds

To identify novel inhibitors of HIV infection, PAK3 modulators described above are typically further tested to confirm their ability to inhibit HIV infection. Many assays and methods are available to examine HIV-inhibiting activity of the PAK3-modulating compounds. This usually involves testing the compounds for ability to inhibit HIV viral replication in vitro or a biochemical activity that is indicative of HIV infection. For example, effect of the PAK3-modulating compounds on HIV infection can be monitored in a PAK3-expressing cell in vitro. Typically, the cell expresses PAK3 endogenously, e.g., HeLa-CD4-Bgal cell as exemplified in the Examples below.

In some methods, potential inhibitory activity of PAK3-modulating compounds on HIV infection is tested by examining their effect on HIV infection of a cultured cell line in vitro. For example, the compounds can be tested on HIV infection of a primary macrophage culture as described in Seddiki et al., AIDS Res Hum Retroviruses. 15:381-90, 1999. They can also be examined on HIV infection of other T cell and monocyte cell lines as reported in Fujii et al., J Vet Med Sci. 66:115-21, 2004. Additional in vitro systems for monitoring HIV infection have been described in the art. See, e.g., Li et al., Pediatr Res. 54:282-8, 2003; Steinberg et al., Virol. 193:524-7, 1993; Hansen et al., Antiviral Res. 16:233-42, 1991; and Piedimonte et al., AIDS Res Hum Retroviruses. 6:251-60, 1990.

In these assays, HIV infection of the cells can be monitored morphologically, e.g., by a microscopic cytopathic effect assay (see, e.g., Fujii et al., J Vet Med Sci. 66:115-21, 2004). It can also be assessed enzymatically, e.g., by assaying HIV reverse transcriptase (RT) activity in the supernatant of the cell culture. Such assays are described in the art, e.g., Reynolds et al., Proc Natl Acad Sci U S A. 100:1615-20, 2003; and Li et al., Pediatr Res. 54:282-8, 2003. Other assays monitor HIV infection by quantifying accumulation of viral nucleic acids or viral antigens. For example, Winters et al. (PCR Methods Appl. 1:257-62, 1992) described a method which assays HIV gag RNA and DNA from HIV infected cell cultures. Vanitharani et al. described an HIV infection assay which measures production of viral p24 antigen (Virology 289:334-42, 2001). Viral replication can also be monitored in vitro by a p24 antigen ELISA assay, as described in, e.g., Chargelegue et al., J Virol Methods. 38(3):323-32, 1992; and Klein et al., J Virol Methods. 107(2):169-75, 2003. All these assays can be employed and modified to assess anti-HIV activity of the PAK3-modulating compounds of the present invention.

In some other embodiments, HIV infection is monitored by examining expression of a reporter gene under the control of HIV-LTR in HIV-infected cells. As exemplified in the Examples below, by measuring expression of a reporter protein (e.g., beta-galactosidase), such assays can monitor any modulating activity on HIV infection. Similar assays have been described in the art. For example, Gervaix et al. (Proc Natl Acad Sci USA. 94:4653-8, 1997) developed a stable T-cell line expressing a plasmid encoding a humanized green fluorescent protein (GFP) under the control of a HIV-I LTR promoter. Upon infection with HIV-I, a 100- to 1,000-fold increase of fluorescence of infected cells can be observed as compared with uninfected cells. These in vitro systems enable monitoring of the PAK3-modulating compounds on HIV infection in real time, quantitation of infected cells over time, and also allow easy and accurate determination of susceptibility to the compounds.

VII. Therapeutic Applications

By inhibiting HIV infection, the PAK3-modulating compounds described above provide useful therapeutic applications of the present invention. They can be readily employed to prevent or treat HIV infections, as well as diseases or conditions associated with HIV infections (e.g., AIDS) in various subjects. HIV infections that are amenable to treatment with the PAK3-modulating compounds of the invention encompass infection of a subject, particularly a human subject, by any of the HIV family of retroviruses (e.g., HIV-I, HIV-II, or HIV-III). The PAK3-modulating compounds are useful for treating a subject who is a carrier of any member of the HIV family of retroviruses. They can be used to treat a subject who is diagnosed of active AIDS. The compounds are also useful in the treatment or prophylaxis of the AIDS-related conditions in such subjects. Subjects who have not been diagnosed as having HIV infection but are believed to be at risk of infection by HIV are also amenable to treatment with the PAK3-modulating compounds of the present invention.

Subjects suffering from any of the AIDS-related conditions are suitable for treatment with the PAK3-modulating compounds. Such conditions include AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome, etc.

Standard methods for measuring in vivo HIV infection and progression to AIDS can be used to determine whether a subject is positively responding to treatment with the Pak3-modulating compounds of the invention. For example, after treatment with a PAK3-modulating compound of the invention, a subject's CD4⁺ T cell count can be monitored. A rise in CD4⁺ T cells indicates that the subject is benefiting from administration of the antiviral therapy. This, as well as other methods known to the art, may be used to determine the extent to which the compounds of the present invention are effective at treating HIV infection and AIDS in a subject.

The PAK3 modulators of the present invention can be directly administered under sterile conditions to the subject to be treated. The modulators can be administered alone or as the active ingredient of a pharmaceutical composition. The therapeutic composition of the present invention can also be combined with or used in association with other therapeutic agents. In some applications, a first PAK3 modulator is used in combination with a second PAK3 modulator in order to inhibit HIV infection to a more extensive degree than cannot be achieved when one PAK3 modulator is used individually. In some other applications, a PAK3-modulating compound of the present invention may be used in conjunction with known anti-HIV drugs such as AZT.

Pharmaceutical compositions of the present invention typically comprise at least one active ingredient together with one or more acceptable carriers thereof. Pharmaceutically acceptable carriers enhance or stabilize the composition, or facilitate preparation of the composition. Pharmaceutically acceptable carriers are determined in part by the particular composition being administered (e.g., nucleic acid, protein, or modulatory compounds), as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, intravenous, or parenteral. For example, the PAK3-modulating compound can be complexed with carrier proteins such as ovalbumin or serum albumin prior to their administration in order to enhance stability or pharmacological properties.

The pharmaceutical compositions can be prepared in various forms, such as granules, tablets, pills, suppositories, capsules, and the like. The concentration of therapeutically active compound in the formulation may vary from about 0.1 100% by weight. Therapeutic formulations are prepared by any methods well known in the art of pharmacy. The therapeutic formulations can be delivered by any effective means which could be used for treatment. See, e.g., Goodman & Gilnan's The Pharmacological Bases of Therapeutics, Hardman et al., eds., McGraw-Hill Professional (10^{th} ed., 2001); Remington: The Science and Practice of Pharmacy, Gennaro, ed., Lippincott Williams & Wilkins (20^{th} ed., 2003); and Pharmaceutical Dosage Forms and Drug Delivery Systems, Ansel et al. (eds.), Lippincott Williams & Wilkins (7^{th} ed., 1999).

The therapeutic formulations can be conveniently presented in unit dosage form and administered in a suitable therapeutic dose. A suitable therapeutic dose can be determined by any of the well known methods such as clinical studies on mammalian species to determine maximum tolerable dose and on normal human subjects to determine safe dosage. Except under certain circumstances when higher dosages may be required, the preferred dosage of a PAK3 modulator usually lies within the range of from about 0.001 to about 1000 mg, more usually from about 0.01 to about 500 mg per day.

The preferred dosage and mode of administration of a PAK3 modulator can vary for different subjects, depending upon factors that can be individually reviewed by the treating physician, such as the condition or conditions to be treated, the choice of composition to be administered, including the particular PAK3 modulator, the age, weight, and response of the individual subject, the severity of the subject's symptoms, and the chosen route of administration. As a general rule, the quantity of a PAK3 modulator administered is the smallest dosage which effectively and reliably prevents or minimizes the conditions of the subjects. Therefore, the above dosage ranges are intended to provide general guidance and support for the teachings herein, but are not intended to limit the scope of the invention.

### Examples

The following examples are offered to illustrate, but not to limit the present invention.

### Example 1

### PAK3 is Involved in HIV Infection

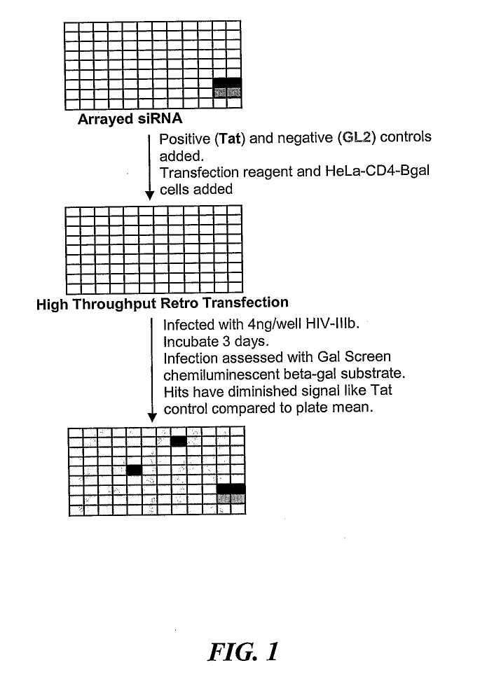

In order to find new host factors involved in HIV infection, we performed a screen of a focused siRNA library (Qiagen) using HeLa-CD4-Bgal cells (Kimpton et al., J Virol 66:2232-2239, 1992). The HeLa-CD4-Bgal cells were obtained from Dr. Michael Emerman through the AIDS Research and Reference Reagent Program, Division of AIDS, NIAID, NIH. The siRNA library was directed against 5000 different genes that have the most potential to be drug targets, with each gene represented by two different siRNAs. As illustrated in FIG. 1, the cells were transfected using a reverse transfection protocol with siRNA against Tat used as a positive control and were challenged with HIV-IIIb 24 hours after transfection (Huang et al., Proc. Nat. Acad. Sci U.S.A. 101:3456-61, 2004). Infection was allowed to proceed for 3 days to allow for effects on all stages of infection from entry to release and spread throughout the culture to be seen. By monitoring reporter gene expression in the HeLa-CD4-Bgal cells, this system allows one to detect any modulating effect of the siRNAs on HIV infection.

TABLE 1
siRNA screen results

| Gene | afa.mfa |
|---|---|
| TSG101 - 1 | −6.61 |
| TSG101 - 2 | −3.15 |
| Furin - 1 | −2.31 |
| Furin - 2 | −3.80 |
| CXCR4 | −2.75 |
| PAK3 | −2.86 |
| PAK5 | −2.51 |

Infection was assessed by measuring the amount of beta-galactosidase produced off the viral LTR promoter using a chemiluminescent substrate. The entire screen was conducted in duplicate and the data was expressed as a ratio of average fold activation (afa) to adjusted standard deviation of the fold activation (mfa), a value that takes into account both the effect of each siRNA and the deviation between the replicates. For those genes whose afa was less than 1 (inhibitors of infection), values were converted to negative fold activation.

As shown in Table 1, many siRNAs against genes already known to be involved in HIV infection, such as CXCR4, TSG101 and furin, were identified as inhibitors by the screen and thus served as internal controls. For TSG101 and furin, both siRNAs designed to target the protein were effective. In addition, two PAK kinases, PAK3 and PAK5 (also known as PAK7), were identified as being involved in HIV infection in this system. While less is known about PAK5, PAK3 is a member of the Group I PAK kinases, which also includes PAK1 and PAK2. Both PAK1 and PAK2 have been shown to be able to associate with Nef leading to enhanced viral production (Fackler, et al., Mol. Cell. Biol. 20:2619-27, 2000; and Renkema et al., Curr. Biol. 9:1407-10, 1999). While the strongest evidence points to PAK2 as being the dominant PAK involved in enhancement of infection, none of the previous studies has used siRNA to determine the PAK kinases specifically involved in physiologic HIV infection.

The identification of PAK3 in this siRNA screen indicates that PAK3 plays an important role in HIV infection. The decreased beta-galactosidase activity in the HIV-infected HeLa-CD4-Bgal cell could be due to a defect at any stage of HIV infection cycle, e.g., viral gene expression, upstream events such as viral entry, reverse transcription, and integration, or downstream activities such as viral RNA export, particle formation, and particle release. Thus, it is not clear from this system how PAK3 down-regulation negatively modulates HIV infection.

### Example 2

### Knockdown of Endogenous PAK3 Inhibits HIV Infection

We obtained siRNA against PAK1 (PAK1-0 and PAK1-3), PAK2 (siGenome pool from Dharmacon) and PAK3 (PAK3-2 and PAK3-3). Each siRNA was validated in HeLa-CD4-Bgal cells by western blot. Both siRNAs against PAK1 effectively knocked down the protein, with PAK1-0 being more effective than PAK1-3. The validated Dharmacon pool siRNA against PAK2 was also very effective, with near total protein knockdown by 72 hours post-transfection. As with the PAK1 siRNAs, both PAK3 siRNAs were effective, with PAK3-2 being more effective than PAK3-3. There was no cross reactivity between the PAK isoform siRNAs.

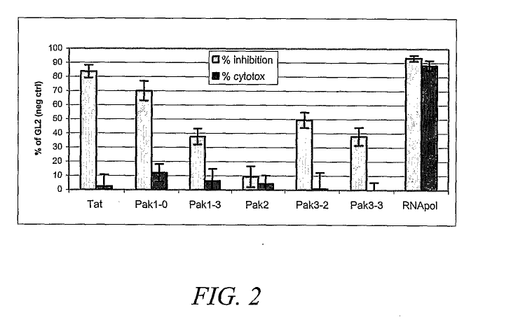

Activity of the siRNA on HIV infection in HeLa-CD4-Bgal cells was examined as described in Example 1. As shown in FIG. 2, the relative efficacy of the PAK1 and PAK3 siRNAs at protein knockdown was reflected in their ability to inhibit HIV infection in HeLa-CD4-Bgal cells. PAK1-0 was almost as effective at blocking HIV as the Tat siRNA control, inhibiting by ˜70% while PAK1-3 was less effective, blocking by ˜38%. PAK3-2, which was the siRNA identified in the screen, continued to block infection by ˜50%, with the less effective PAK3-3 showing less inhibition (˜38%). Contrary to what is suggested in the current literature, siRNA against PAK2 had little to no effect on HIV infection despite being able to effectively knockdown protein levels. None of the siRNAs tested had significant cytotoxicity in the HeLa cells (FIG. 2), ruling out overall toxicity as a mechanism for infection inhibition. Together, these results suggest a significant role of PAK1 and PAK3 in HIV infection of HeLa-CD4-Bgal cells and argue against a major role for PAK2.

In order to investigate the effects of Group I PAK siRNAs in a more physiologic cell line target they were tested in Jurkat cells. Jurkat cells were electroporated with the most effective siRNA against PAK1 and PAK3 or with the PAK2 pool. As seen in the HeLa-CD4-Bgal cells, PAK1 knockdown resulted in inhibition of HIV infection while PAK2 knockdown had little to no effect despite the specific knockdown of protein levels. The lower level of inhibition in Jurkats with PAK1 siRNA compared to HeLa-CD4-βgal cells is reflected by the lower level of protein knockdown seen. None of the siRNAs had any cytotoxicity as shown by Cell Titer Glo, again eliminating cytotoxicity as the reason for inhibition. PAK3 knockdown had no effect on infection, a result consistent with the lack of expression of PAK3 in this cell line. This may highlight the involvement of different PAK kinases in HIV infection depending on the tissue type being infected. The primary site of PAK3 expression is the brain, where HIV actively replicates soon after seroconversion (Bissel et al., Brain Pathol. 14:97-108, 2004; and Pomerantz et al., DNA Cell Biol. 23:227-38, 2004.). These results indicate that while PAK1 may be the dominant PAK involved in T-cell infection, infection in the brain can also involve PAK3.

It is understood that the examples and embodiments described herein are for illustrative purposes only and that various modifications or changes in light thereof will be suggested to subjects skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, patents and patent applications cited herein are hereby expressly incorporated by reference in their entirety and for all purposes as if each is individually so denoted.

We claim:

1. A method for identifying an agent that inhibits HIV infection, comprising assaying a biological activity of a p21-activated kinase 3 (PAK3) molecule or a fragment thereof, in the presence of test compounds to identify a compound that inhibits the biological activity of the PAK3 molecule; thereby identifying an agent that inhibits HIV infection.

2. The method of claim 1, further comprising testing the identified compound for ability to inhibit HIV infection.

3. The method of claim 1, wherein the biological activity is the kinase activity of the PAK3 molecule or the expression of a gene encoding the PAK3 molecule.

4. The method of claim 1, wherein the PAK3 molecule is derived from a mammalian cell.

5. The method of claim 4, wherein the PAK3 molecule is human PAK3.

6. The method of claim 2, wherein the ability to inhibit HIV infection by the compound is examined by monitoring expression of a reporter gene under the control of HIV LTR promoter in an HIV-infected cell.

7. The method of claim 6, wherein the cell is HeLa-CD4-Bgal.

8. The method of claim 6, wherein the reporter gene is a beta-galactosidase gene.

9. The method of claim 2, wherein the ability to inhibit HIV infection by the compound is examined by monitoring HIV replication in a PAK3-expressing cell in vitro.

10. The method of claim 9, wherein HIV replication is monitored via a p24 antigen ELISA assay or a reverse transcriptase activity assay.

11. The method of claim 9, wherein the cell is a primary macrophage cell.

12. A method for identifying an agent that inhibits HIV infection, comprising (a) screening test compounds to identify PAK3-modulating compounds that down-regulate the kinase activity or cellular level of a PAK3 molecule, and (b) testing the identified PAK3-modulating compounds for ability to inhibit HIV infection.

13. The method of claim 12, wherein the PAK3 molecule is derived from a mammalian cell.

14. The method of claim 13, wherein the PAK3 molecule is human PAK3.

15. The method of claim 12, wherein the ability to inhibit HIV infection by the compounds is examined by monitoring expression of a reporter gene under the control of HIV LTR promoter in an HIV-infected cell.

16. The method of claim 15, wherein the cell is HeLa-CD4-Bgal.

17. The method of claim 15, wherein the reporter gene is a beta-galactosidase gene.

18. The method of claim 12, wherein the ability to inhibit HIV infection by the compounds is examined by monitoring HIV replication in a PAK3-expressing cell in vitro.

19. The method of claim 18, wherein HIV replication is monitored via a p24 antigen ELISA assay or a reverse transcriptase activity assay

20. The method of claim 18, wherein the cell is a primary macrophage cell.

21. A method for treating HIV infection in a subject, the method comprising administering to the subject a pharmaceutical composition comprising an effective amount of a PAK3-modulating compound which down-regulates the kinase activity or the expression of a p21-activated kinase 3 (PAK3); thereby treating HIV infection in the subject.

22. The method of claim 21, wherein the PAK3 is a human PAK3.

23. The method of claim 21, wherein the PAK3-modulating compound inhibits HIV replication in a PAK3-expressing cell in vitro.

24. The method of claim 23, wherein the cell is HeLa-CD4-Bgal.

25. The method of claim 21, wherein the PAK3-modulating compound is administered to the subject concurrently with a known anti-HIV drug.
